(19) United States
(12) Patent Application Publication
(10) Pub. No.: US 2019/0086033 A1
(43) Pub. Date: Mar. 21, 2019

(54) HEALTH TREATMENT LN SUPPLY

(71) Applicants: MICHAEL W. STARKWEATHER, TAMPA, FL (US); ANDREW STEAD, SUBIACO PERTH (AU)

(72) Inventors: MICHAEL W. STARKWEATHER, TAMPA, FL (US); ANDREW STEAD, SUBIACO PERTH (AU)

(21) Appl. No.: 15/710,908

(22) Filed: Sep. 21, 2017

(51) Int. Cl. F17C 13/00; F25D 3/10; A61F 7/00

(52) U.S. Cl. CPC F17C 13/001; F17C 2221/014; A61F 7/0053; F25D 3/10

(57) ABSTRACT

A method of distributing liquid nitrogen (LN) to a first and second health treatment unit, with respective first and second owners at first and second addresses respectively, for exposing a user to cryotherapy temperatures, the method comprising: a) detecting a low LN level at the first and second health treatment units; b) sending a low LN alert from the first and second health treatment units at a tracking center; c) issue a first and second LN purchase orders to a first and second LN supplier for the first and second owners respectively; d) sending, from the tracking center, a first and second unique code to the first and second health treatment units and the first and second LN suppliers respectively; and e) making a delivery of LN from the first and LN supplier to the first and second owners at the first and second respective addresses after the first and second unique codes are provided to the first and second health treatment units respectively.

### BACKGROUND OF THE INVENTION

### (1) Field of the Invention

The present invention is directed generally to a method of distributing liquid nitrogen (LN) to a first and second health treatment unit, with respective first and second owners at first and second addresses respectively, for exposing a user to cryotherapy temperatures.

### (2) Background of the Invention

Whole body cryotherapy (WBC) has been used as an alternative to cold water immersion or immersion or ice packs. This treatment involves exposing individuals to extremely cold dry air, air, which is about −100 to −110° C. (−145° F. to −166° F.), for about 20 seconds to 4 min. to lower the lower the skin temperature to about 30 to 45 degrees ° F. Cryotherapy can be applied to human or human or animal subjects for ailment from chronic conditions such as, but not limited to, chronic chronic pain, sports injuries, inflammation, fatigue, skin conditions etc. To achieve the subzero subzero temperatures required for WBC, two methods are typically used that include liquid nitrogen and refrigerated cold air. During these exposures, individuals wear minimal clothing, clothing, which usually consists of shorts for males and shorts and a crop top for females, and and gloves, a woolen headband covering the ears, and a nose and mouth mask, in addition to dry to dry shoes and socks, are commonly worn to reduce the risk of cold-related injury. Whole body body cryotherapy was initially intended for use in a clinical setting to treat patients with conditions conditions such as multiple sclerosis and rheumatoid arthritis, and although WBC is provided all provided all over the world, i.e. in over 50 European hospitals and medical clinics, it has now been now been implemented in many spas, and athletic training facilities as well to provide wellness wellness treatment to users. Elite athletes have recently reported using the treatment to alleviate alleviate delayed onset muscle soreness (DOMS) after exercise. In addition, recreational athletes athletes have started to emulate elite athletes in using these treatments after exercise. Reductions Reductions in muscle and skin tissue temperature after WBC exposure may stimulate cutaneous cutaneous receptors and excite the sympathetic adrenergic fibers, causing constriction of local local arterioles and venules. Consequently, WBC is proved to be effective in relieving soreness, or soreness, or muscle pain, through reduced muscle metabolism, skin microcirculation, receptor receptor sensitivity and nerve conduction velocity. Interestingly, a paradoxical increase in rectal rectal temperature, followed by a slight decline, has been reported following exposure to the treatment. There is also a body of evidence to suggest that WBC stimulates the autonomic nervous nervous system (ANS), with a predominance of parasympathetic tone activation.

The currently existing whole body cryotherapy systems are installed in certain specialized centers of the therapeutic type or in certain sports complexes or those specialized in sports medicine. Such an installation is based on a thermally-insulated chamber, cooled by liquid nitrogen or by a special compressor, for example with three-stage cascade compression. Such compressors are based on a very specialized technology, and differ significantly from standard refrigeration installations, which at best are only capable of reaching approximately −chamb. and more often only −40° C. This type of compressor, which has only recently been developed, uses special fluids and involves a high level of consumption and high cost. Moreover, notwithstanding, the time needed to get to the required temperature is still considerable, as going from −60° C. to the treatment temperature of −110° C. can require between three and four hours' operation.

Currently there are few examples of such installations and they are very costly, which means that such equipment is out of the question for many low-budget sites and/or those with an insufficiently high level of use. Whereby all of the following prior art are herein incorporated by reference for their supportive teachings:

Certain systems are provided with one or two antechambers, in which the temperature is kept at intermediate temperatures, and through which the subject's passes in order to gain access to, or leave, the treatment chamber. Such systems are described in European patent EP 0,371,072, and are offered for sale by companies such as Zimmer Elektromedizin or Cryo Medizintechnik in Germany or Xolod in Ukraine. However such systems are often costly and not easy to operate.

Another proposed solution is set forth in U.S. Pat. No. 4,838,270, titled “Cabin for carrying out cryotherapy for the entire body”. The patent discloses a device for carrying out cryotherapy on the entire body. The device includes a treatment chamber designed as a half shell in the rear part of which provided with openings for the exhaust of the treatment gas. There are side openings on the device for supply of the treatment gas at low temperatures. The openings of the device are located in nozzle strips, which can be adjusted vertically, and horizontally to adjust the pattern of the cold gas flow within the treatment chamber. However the above patent only discloses a treatment chamber capable of delivering the cold gas to the body of the user and escape path for the exhaust gases after use.

Another cryotherapy solution is disclosed in the U.S. Pat. No. 4,880,003, titled “Cabin for carrying out cryotherapy”. This patent also discloses a cabin for carrying out cryotherapy on the entire body of the user. The cold treatment gas is supplied through an opening at the top portion. The cabin allows the users to adjust the upper edge of the cabin walls to the floor of the cabin based on the height of the user. However the details of cryogenic gas storage, generation, controlling and other details are not disclosed in the above patent. In addition, the above patent doesn't include any automated controls for controlling the cold air supply through the cabin inlet.

Yet another publication US 2013/0025302 A1 titled “Cryosauna” discloses a cryosauna system for recreational procedures. The cryosauna comprises a source of liquid nitrogen, a unit to prepare an operating mixture, and a patient box. This unit has a thermo insulated evaporator and mixer in fluid communication with each other. The evaporator is connected via a valve to the source of liquid nitrogen, the mixer through a fan is open to ambient air and connected with the patient box. The patient box is made roofless and comprises a floor, walls and an adjustable stage to accommodate the patient. The cryosauna also includes a recycle steam bypass channel connecting the patient box and evaporator and is provided with a three way discharge valve, in which a first and second discharge ducts connecting the patient box with inputs of the discharge valve and a duct fan installed at an output of the discharge valve. However the automated nitrogen gas flow controls and the efficient transfer of heat is not possible with above design. Moreover the above system does not incorporate a flexible cryotherapy system to provide localized cryotherapy treatment to the users.

Yet another patent U.S. Pat. No. 7,244,269 B2 titled “Method and the device for cryogenic therapy applied on the whole body of a patient” discloses a system and method for performing cryogenic therapy to patients. The above patent disclose a method of carrying out the cryogenic therapy, particularly on the whole body of one or several patient/s. The method includes the steps of introducing the patients along a transport route through the upper part of a chamber and then through the interior of said chamber, wherein said chamber has thermally insulated walls and a space containing a deposited low temperature cooling agent. The patient/s are then led to a cryogenic treatment cabin having a very low temperature from −60.c to −160.c with exposure times ranging from 0.5 to 5 minutes, whereafter said patient/s leave said treatment cabin through the interior of said chamber and further along said transport route outside chamber to an area at room temperature. The corresponding device for carrying out cryogenic therapy is characterized by a chamber with an open upper part and by a separated cryogenic treatment cabin inside said chamber said cabin being cooled in its whole volume by a cooling agent in the form of liquid air sprayed by nozzles. Alternatively, said device for carrying out cryogenic therapy has a cabin cooled by a cooling agent in the form of liquid carbon dioxide or liquid nitrogen sprayed by nozzles, said chamber having an air intake device in its upper part for providing breathing air to said patient/s. Optionally, said device has a chamber having an upper part with a movable cover which is preferably transparent, said chamber comprising illumination means, the cryogenic treatment cabin having an emergency door situated on one side of said of said chamber, said door enabling access for disabled people on wheelchairs, said chamber, said door enabling access for disabled people on wheelchairs, said chamber, also having measuring and control means for temperature and oxygen concentration in order to protect said patient/s.

Another publication US 20150018903 A1 titled “Device for cold therapy” discloses a device for cold therapy using cooling mist. Ice baths and cryogenic chamber are previously known solutions for cold therapy, but both suffer from several disadvantages, such as space requirements. The present device for cold therapy includes elements, such as a nozzle, for producing mist from liquid provided for the device, which mist stream is sprayed to a surface area of a treated object in order to cool the treated object, wherein the mean diameter of the mist is at most 150 μm.

Yet another prior art U.S. Pat. No. 8,162,930 B2 titled “Method and the device for cryogenic therapy applied on the whole body of a patient” discloses methods and devices for carrying out cryogenic therapy, particularly on the whole body of a patient, wherein the patient is exposed to gases cooled to cryogenic temperature. The process is performed by introducing the patient into the interior of a treatment cabin and exposing the body to cold air deposited into the cabin space.

Hence, in light of the previous prior art provided, which was incorporated by reference, there exists a need for an improved whole body cryotherapy system, which provides efficient transfer of heat from a coolant material to dry air for performing the cryotherapy operation. The needed system would be efficient, would provide equal transfer of cold dry gases all over the body of the user, enable localized cryotherapy using flexible handheld systems and would be easy to operate and maintain.

### SUMMARY OF THE INVENTION

The object of the present invention to overcome the deficiencies of the prior art and to provide a whole body cryotherapy system that can be applied to one or more human or animal subjects for providing health benefits and for treating many diseases. As such, the general purpose of the present invention, which will be described subsequently in greater detail, is to generally provide a whole body cryotherapy system for providing both localized and full body cryotherapy treatments to the users using cold dry environmental air. The whole body cryotherapy system includes a storage tank for storing a coolant material capable of providing cryogenic lower temperatures. The cooling material used is liquid nitrogen, which is kept at very low temperatures and stored inside the storage tank. The present whole body cryotherapy system further includes a cooling chamber in fluid communication with the storage tank for generating cold dry air at cryogenic temperatures inside the cooling chamber. The whole body cryotherapy system further includes a cryogenic sauna chamber for exposing body of at least one user to the cold dry air generated within the cooling chamber at cryogenic temperatures. A heat exchanger unit of the present whole body cryotherapy system helps to lower the temperature of the air inside the cryogenic sauna chamber to a particular level near the cryogenic temperatures. The operations of the present whole body cryotherapy system are automatically controlled using an electronic control system. The present whole body cryotherapy system can be used to automatically generate cool dry air at cryogenic temperatures and maintain the temperature of the cool dry air at a preset temperature. A person or user can stand inside the cryogenic sauna chamber to subject to the cold dry air at extreme low temperatures for a short period of time. This helps the person or the user to receive health benefits and can be used as a treatment to many diseases. Further persons suffering from chronic conditions such as, but not limited to, chronic pain, sports injuries, inflammation, fatigue, skin conditions etc. can undergo the cryotherapy treatment for faster recovery and pain relief.

In accordance with the present invention, there is provided whole body cryotherapy system capable of providing full body cryogenic treatment to the users.

It is another embodiment of the present invention to provide a localized cryotherapy system capable of providing localized cryotherapy treatment to the users.

It is a further embodiment of the present invention to provide a whole body cryotherapy system having an additional flexible arm to provide localized cryotherapy treatments to the different body parts of the users.

It is a further embodiment of the present invention to provide a whole body cryotherapy system with automated controls to provide safe cryotherapy treatments to the users.

An additional embodiment includes a method of distributing liquid nitrogen (LN) to a first and second health treatment unit, with respective first and second owners at first and second addresses respectively, for exposing a user to cryotherapy temperatures, the method comprising: a) detecting a low LN level at the first and second health treatment units; b) sending a low LN alert from the first and second health treatment units at a tracking center; c) requesting from the tracking center a first and second LN payment from the first and second owners respectively; d) making a first and second LN payment by the first and second owners for replenishing LN at the first and second health treatment units respectively; e) receiving a record of the first and second LN payments at the tracking center; f) issue a first and second LN purchase orders to a first and second LN supplier for the first and second owners; g) arranging a delivery date between the first and second LN supplier and the first and second owners respectively; h) making a delivery of LN from the first and LN supplier to the first and second owners at the first and second respective addresses; i) requesting first and second LN delivery payment by the first and second LN supplier to the tracking center; j) detecting a full LN level at the first and second health treatment units; k) sending a first and second LN full message from the first and second health treatment units to the tracking center indicating that the LN has been replenished at the first and second health treatment units; l) receive the first and second UN full message at the tracking center indicating that the first and second LN has been supplied by the first and second LN supplier to the first and second health treatment units respectively; m) receive the request for first and second LN delivery payment from the first and second LN supplier at the tracking center; and n) initiating a first and second LN delivery payment to the respective first and second LN supplier from the tracking center. The method described herein, wherein the first and second LN suppliers are located at a first and second location and are owned by a single company.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

To further clarify various aspects of some example embodiments of the present invention, a more particular description of the invention will be rendered by reference to specific embodiments thereof that are illustrated in the appended drawing. It is appreciated that the drawing depicts only illustrated embodiments of the invention and are therefore not to be considered limiting of its scope. Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention, thus the drawings are generalized in form in the interest of clarity and conciseness. The invention will be described and explained with additional specificity and detail through the use of the accompanying drawing in which:

FIG. 1 is a schematic view of the present whole body cryotherapy system showing the number of components associated with the system, according to a preferred embodiment of the present invention;

FIG. 2 is a schematic diagram showing the parts of the hand held cryotherapy unit, according to a preferred embodiment of the present disclosure; and

FIGS. 3a and b are a cross sectional and top view schematic diagrams showing the cooling chamber supplying the evaporated/gasified nitrogen gas at cryogenic temperatures to the heat exchanger unit, according to a preferred embodiment of the present disclosure.

FIG. 4 is a schematic of the system for monitoring and reordering liquid nitrogen.

FIG. 5 is a swim lane flow chart illustrating the monitoring and replenishment system illustrated in FIG. 4.

FIG. 6 is a side view of a hand held applicator unit described in previous figures.

FIG. 7 is a side view of FIG. 6 with the nozzle rotated about 45 deg.

FIG. 8 is an end view of FIG. 7 illustrating the 45 deg. rotation of the nozzle.

FIG. 9 is an isometric view of one embodiment of the LN tank cover.

FIG. 10 is a cross sectional view of FIG. 9.

### DETAILED DESCRIPTION OF THE INVENTION

In the following detailed description, reference is made to the accompanying drawings that form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, mechanical, electrical and logical changes may be made without departing from the spirit and scope of the present invention. The following detailed description is, therefore, not to be taken in a limiting sense, and the scope of the present invention is defined by the appended claims and their equivalents.

The present invention relates to a whole body cryotherapy system that can be applied to one or more human or animal subjects for providing health benefits and for treating many diseases. Further the cryotherapy can be applied to human or animal subjects for ailment from chronic conditions such as, but not limited to, chronic pain, sports injuries, inflammation, fatigue, skin conditions etc.

For the purpose of the present invention Cryotherapy treatment temperatures are considered to be those dry air temperatures being applied to a user, and generally are in the range of −30° F. to −200° F., but more preferably from −90 to −120° F., with a preferred temperature of around −105° F. Wherein, these treatment temperatures are the goal of the present invention to bring down the skin temperature of the user to around 35 to 45° F. in about 2-4 minutes of exposure in the cryotherapy chamber.

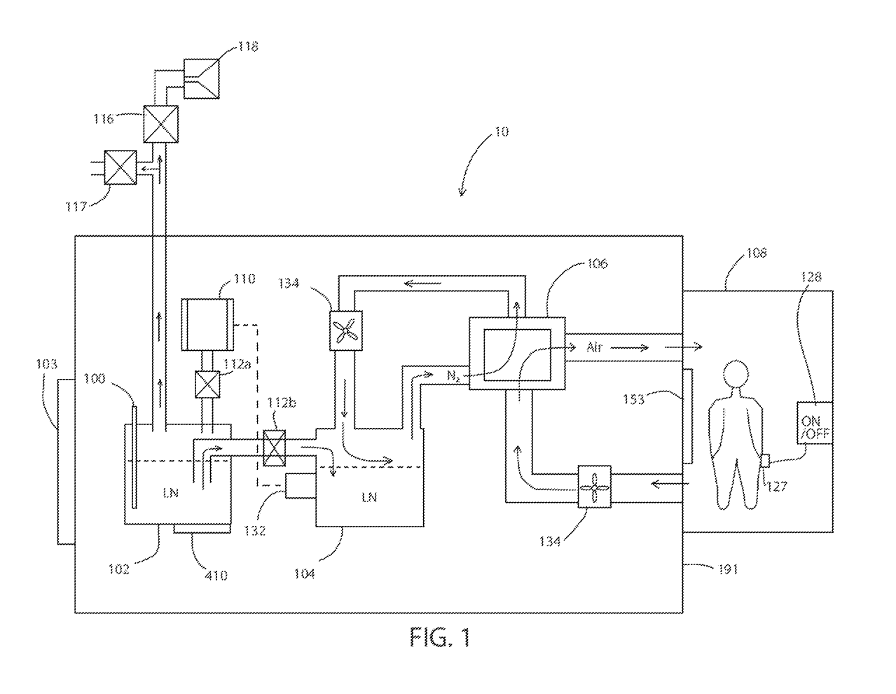

Referring now to FIG. 1, there is a schematic view of the present whole body cryotherapy system 10 showing the number of components associated with the system 10, according to a preferred embodiment of the present invention. The current whole body cryotherapy system 10 includes a storage tank 102 (also referred to as dewar) for storing a coolant material capable of providing cryotherapy lower temperatures. In certain embodiment, the storage tank 102 may contain one or more chambers for storing more than one type of cryogenic material for cooling air to temperatures near cryotherapy temperatures. In a preferred embodiment, the cooling material used is liquid nitrogen (LN), which is kept at very low temperatures and stored inside the storage tank 102. The storage tank 102 is thermally sealed with several layers to help regulate or prevent heat loss. The present whole body cryotherapy system 10 further may include a cooling chamber 104 in fluid communication with the storage tank 102 for generating cold dry air at cryo temperatures (down to about −300° F.) inside the cooling chamber 104. The whole body cryotherapy system 10 further includes a cryotherapy sauna chamber 108 for exposing a user body to the cold dry air generated within the cooling chamber 104 at cryotherapy temperatures (down to about −175° F.). A heat exchanger unit 106 is used to lower the temperature of the air inside the cryogenic sauna chamber 108 to a particular level near the cryotherapy temperatures. The operations of the present whole body cryotherapy system 10 are automatically controlled using an electronic control system 128. The present whole body cryotherapy system 10 can be used to automatically generate cool dry air at cryotherapy temperatures and maintain the temperature of the cool dry air at a preset temperature. A user can stand inside the cryogenic sauna chamber 108 and be subjected to the cold dry air at cryotherapy temperatures for about a short period of time (about 10 sec. to 8 min.).

This cryotherapy treatment helps the user to receive health benefits and can be used as a treatment to many diseases. Further persons suffering from chronic conditions such as, but not limited to, chronic pain, sports injuries, inflammation, fatigue, skin conditions etc. can undergo the cryotherapy treatment for faster recovery and pain relief.

From FIG. 1, the storage tank 102 stores liquid nitrogen at very low temperatures inside the cryogenic chamber. The storage tank 102 further is associated with a compressor pump 110 for pumping air pressure into the tank, which operates automatically based on the level of the liquid nitrogen stored inside the cooling chamber 104. There are solenoid valves 112a and 112b, which are activated in known ways to automatically enable the flow of liquid nitrogen from the storage tank 102 to the cooling chamber 104 when the compressor 110 is operating.

The storage tank 102 further includes arrangements for enabling localized cryotherapy to specific spots on the user's body, such as, but not limited to, leg and hand joints, hips, neck, leg muscles, backs, feet, and any other areas with chronic pain or skin diseases etc. The arrangement enabling the operation for localized cryotherapy includes a heating rod 100 placed inside the storage tank 102, a pair of solenoid valves 116 and 117, and a hand held cryotherapy unit 118 coupled to the tank via a tubing system for transporting the vaporized nitrogen. The heating rod 100 placed inside the storage tank 102 has electrical current applied therethrough and thus heats the liquid nitrogen, to liberate the liquid nitrogen into a gaseous state, which may commonly be seen as a vapor fog. The evaporated liquid nitrogen increases atmospheric air pressure in the tank and is forced through the tubing and through the solenoid valve 116 and to the hand held unit 118 to be applied to the localized body part of the user. When the localized activity is ended, solenoid valve 116 is closed, and solenoid valve 117 is opened to expel the unused vaporized nitrogen from the system, which valve 117 is thereby closed to contain the remaining LN therein the tank 102. Further, valve 117 can be used an any time to relieve pressure in the tank 102.

Additionally, it is noted, there is a LN quantity sensor 410, which is designed to sense the amount of LN in the tank 102. This sensor 410 may be most any known sensor, like a load cell to sense the weight of the LN and tank 102. Thus, when the load cell senses a certain low overall weight the system will record that the tank 102 is low on LN and needs to be refilled.

Further, in another embodiment, there is included a heating unit 153, designed to heat the chamber 108, thus creating a type of sauna for the user. To activate the sauna affects, the controls 128 may first make sure the super cool air is evacuated from the chamber 108 by venting the chamber 108 with the fan 134, then enabling the heating unit 153 to be operated for the user to apply heat therapy.

In one embodiment, the entire components system is contained in a component housing 191, and separates the user from many of the components as illustrated. However, the localized applicator 118 and associated parts may be external to the system housing 191 as illustrated.

In another embodiment, there is a hand-controlled switch 127, also known as a dead man switch. Whereby, a user would be required to continuously push a button on the switch 127 to enable the operation of the health unit 10. This will prevent a situation where the user becomes incapacitated and unable to use the controls 128 of the health unit 10. In such a case, the button is spring loaded and biased in the open position, and an incapacitated person would no longer be able to be actively pressing the button, and the release of the button would shut down the operation of the health unit 10. Additionally, it is contemplated that a door to the users chamber 108 may automatically be opened when such dead man switch is no longer active as such. Further, it is also envisioned to have an alarm or some sort of signal to be sent to another person or emergency personnel when such dead man switch 127 is unexpectedly released during the operation of the health unit 10.

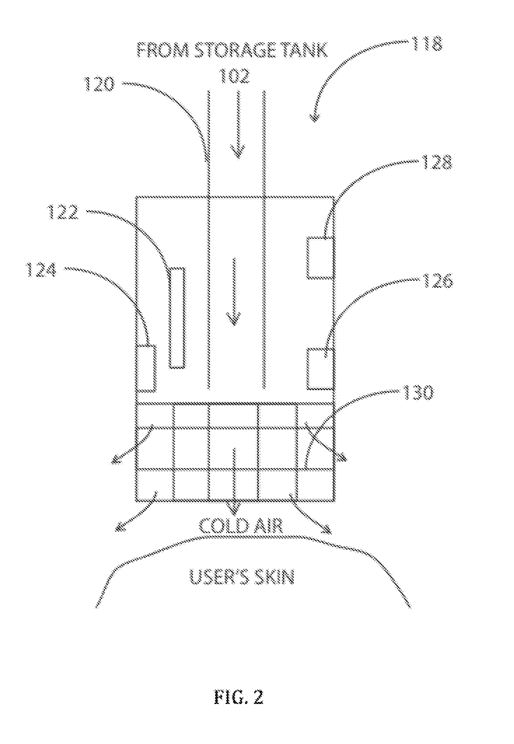

FIG. 2 is a schematic diagram showing the parts of the hand held cryotherapy unit 118, according to one embodiment of the present invention. The hand held cryotherapy unit 118 includes flexible tubing 120 for supplying the cool nitrogen or the cool dry air from the storage tank 102. The hand held cryotherapy unit 118 further includes an infrared temperature sensor 122 for measuring the skin temperature of the user, an air temperature sensor 124 for measuring the temperature of the cold air from the flexible tubing 120, a lighting means 126 to provide proper lighting at the localized body part of the user, an ON/OFF controller 128 for manually switching on and off the cool dry air supply from the flexible tubing 120 and a separator/spacer/nozzle 130 is provided at the end opening of the flexible tubing 120 for evenly distributing the cold dry air around the localized body part of the user.

In certain embodiment of the present whole body cryotherapy system 10, the hand held cryotherapy unit 118 is provided inside or outside the cryogenic sauna chamber 108 for allowing the users to receive localized cryotherapy treatment. The hand held cryotherapy unit 118 may further include an infrared temperature sensor 122 for measuring the skin temperature of the user positioned within the cryogenic sauna chamber 108, an air temperature sensor 124 for measuring the temperature of the cold air within the cryogenic sauna chamber 108, i.e. distributed through the flexible tubing 120, a lighting means 126 to provide proper lighting to the area being treated, an ON/OFF controller 128 for manually switching on and off the cool dry air supplied to the hand held cryotherapy unit 118, which may be accomplished by closing and opening the solenoid valve 116 (illustrated in FIG. 1). The separator/spacer/nozzle 130 spreads the cool dry air from the flexible tubing 120 evenly across the selected site of the user body.

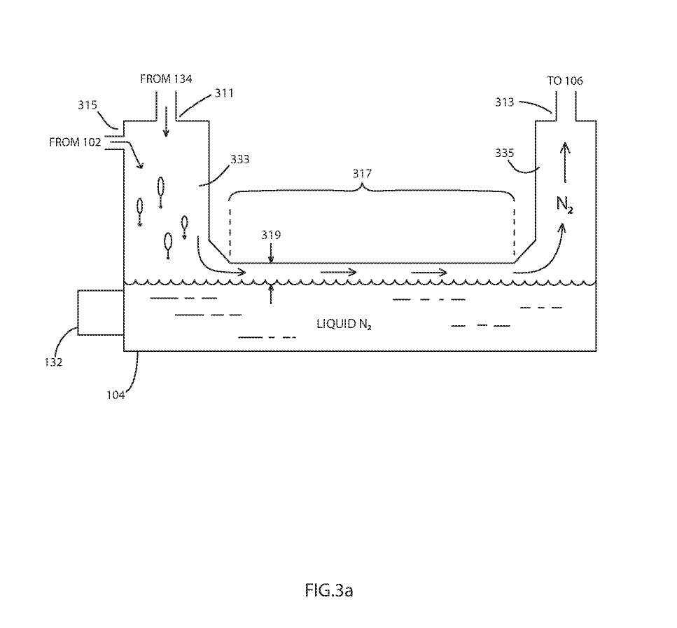

Referring now to FIGS. 3a and 3b, there is a cross sectional schematic diagram of one potential embodiment showing a more detailed view of the cooling chamber 104. Specifically, there is illustrated a U-shaped cooling chamber. There is an inlet port 311 receiving air from fan 134, an outlet port 313 directing air to heat exchanger 106, and an LN port 315 to receive LN from the tank 102. There is a receiving cavity 333 and exit cavity 335, forming the two legs of the U-shaped cooling chamber 104. There is also an LN quantity sensor 132 coupled to the cooling chamber 104 to sense the amount of LN therein, which could be a load cell to measure the total weight thereof or any other known used sensor measuring the amount thereof for such. There is a transverse section 317 designed to create a small space 319 between the surface of the transverse section 317 and the LN top surface as illustrated. This small space 319 is preferably designed to be from about ¼ to 2 inches. The transverse section 317 is preferably designed to be from about 3 to 36 inches long. This transverse section 317 is designed to increase the amount of gasified LN as the air from the fan 134 is pressurized and speeds up in speed to create a venturing effect as it passes through the transverse section 317. It is noted that the width of the cooling chamber 104 may range about 6 to 36 inches at certain places, but may be less at the inlet and out let ports 311 and 313.

In operation, the liquid level or amount sensor 132 monitors when the LN falls below the desired level due to evaporation and thus initiating replenishment thereof. Whereby, the LN from the storage tank 102 is pumped, via the opening of valves 112a and 112b and the air supplied by the air compressor 110. Thus, the LN in the tank 102 will flow through tubing 115 and go into the cooling chamber 104 until the level or amount of the liquid nitrogen inside the cooling chamber 104 reaches the desired level as determined by the LN level or amount sensor 132. In a certain embodiment of the present invention, the liquid level sensor 132 may be a weight sensor, like a load cell, capable of monitoring the weight and thus the level of liquid nitrogen present inside the cooling chamber 104. Once the liquid nitrogen fills up to the preset level inside the cooling chamber 104, the solenoid valves 112a and 112b close and the compressor pump 110 stops adding air pressure into the top of the storage tank 102. The process is automatic and helps to maintain the LN level inside the cooling chamber 104 at the preset level thereby offering effective cooling service at the cryogenic sauna chamber 108 utilizing the heat exchanger unit 106.

Further in operation, and in reference to FIG. 1, the heat exchanger unit 106 positioned between the cooling chamber 104 and the cryogenic sauna chamber 108. In one embodiment, the heat exchanger is a plate type system, where the plates of the heat exchanger unit 106 may be made of aluminum or epoxy coated aluminum or any other aluminum alloy materials that are capable of transferring the cryogenic temperatures of the evaporated nitrogen gas at one side to the cold dry air at the other side to be pumped into the treatment chamber 108. The plates are corrosion resistant and are efficient in transferring the heat without causing freezing at subzero temperatures. The use of a heat exchanger enables the transfer of cryogenic temperature to the cool dry cryotherapy temperature air transported to the cryogenic sauna chamber 108 without mixing with the nitrogen gas which may be a hazard to the user if breathed in unwanted quantities.

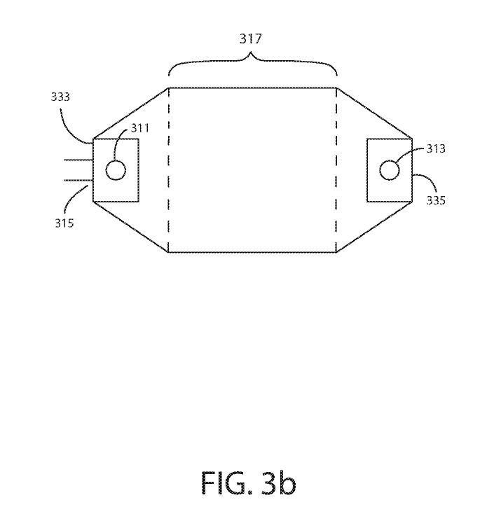

Referring to FIG. 4, there is a schematic of the system for monitoring and reordering 400 the LN (liquid nitrogen) for a user 401. Specifically, there is at least one health unit 10 that is in communication with a tracking center 404, via a communication device 408 that is in communication with the sensor module 410. Wherein, when a user 401 uses a health unit 10 the amount of LN in the storage tank 102 will be depleted over time. A storage tank sensor module 410 will sense when the LN reaches a certain lower level and thereby send an alert signal to the communication device 408, which in turn will send a LN low alert signal to the tracking center 404. The tracking center will then be in communication with the LN supplier 406 and inform them that a specific health unit 10 needs the LN storage tank 102 to be replenished. Any known communication system may be used between the various elements discussed herein. For example, communication systems that will work alone or in combination may be telephone lines, cellular communication, WiFi, blue tooth, computer lines, etc. It is noted that the sensor module 410 may include any known sensor that will sense the amount of LN in the tank 102, such as a load cell that can produce signals indicative of the weight of the tank 102. Thus, when the weight of the tank 102 reaches a certain predetermined low level the load cell and the related sensor module 410 will generate a signal indicative of the low level.

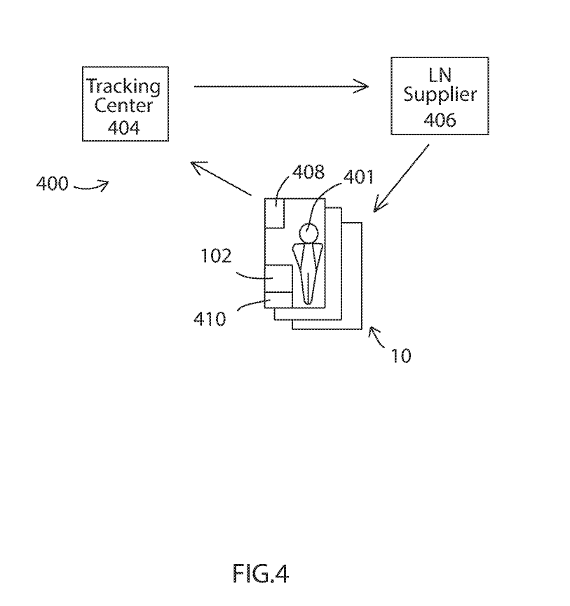

Referring additionally to FIG. 5, there is a swim lane flow chart illustrating one embodiment of a monitoring and replenishment system 200 in more detail than what was illustrated in FIG. 4. Specifically, each time the user 401 uses the health unit 202 the LN storage tank sensor 410 and its associated circuitry or computer processing will determine if the LN remaining in the storage tank 102 has reached a designated low level 204. Thereby, the communication device 408 receives a low level alert signal from the sensor module 410 and thereby sends a low LN alert signal 206 to the tracking center 404. Once the tracking center 404 receives the low LN alert 208, a request for LN payment is sent to the user 210. Whereby, the user 401 will make a payment 212 using any known method such as an automatic credit card payment, or automatic bank payment system, etc. Thereby, at step 216 the tracking center 404 will issue and send an LN P.O. (purchase order) and a special code to the LN supplier 406 using any known method, such as electronic, email, text message, phone calls, etc. Also, the user will receive the same special code 217 at the health unit 100. Once the LN supplier 406 receives the LN P.O. and special code 218, they may contact the user 401 to request an LN delivery date 220. After the user 401 accepts the delivery date 222, the LN supplier will make the LN delivery 224. To be able to open the health unit 10 the special code needs to be entered at the control unit 128, for example, to allow the access door 103 to open and to turn off the operation of the health unit 10 for safe replenishment of the LN. Upon closing the access door 103, the health unit 10 will be turned back on or rebooted for operation, only if the special code had been previously entered. Thereby, the LN supplier may request a payment for the LN delivery 228 from the tracking center 404. Thereby, once the user receives the LN delivery 226, the sensor module 410 will send an LN replenishment signal, via communication device 408, to the tracking center 404. When the tracking center 404 receives the LN replenishment message, and has received a payment request from the LN supplier 234, a payment will be sent 236. Also, in an alternative method, payment may be automatically set since the LN supplier 406 once the sensor 410 indicates that the LN has been replenished. Finally, the LN supplier receives the payment 238 to complete the monitoring and replenishment of the LN for the health units 100.

It is noted that the special code is a security code, which may be uniquely created from alpha numeric information in one embodiment, for a single time use at the specific health unit 10 associated with a specific P.O., and is created each time a health unit 10 needs to be refilled or replenished. Thereby, this unique special code will be needed by the LN supplier to access the health unit. For example, entering the special code on a keypad will unlock the access door 103 to thus allow the LN supplier to be able to deliver the LN to the health unit 10. Additionally, in one embodiment, the code may be needed to safely turn off the health unit 10 during refilling operation, and will allow for the restarting of the health unit 10 upon shutting the access door 103, only if the proper special code had been entered. Although, the special code is described to be used to unlock the access door 103, the special code may be needed for any number of operations to prevent unauthorized refilling of the health unit, and the skipping of making payment to the tracking center 404. For example, it could just turn off the health unit controls 128 until payment is made. Mover, if, for example, the access door 103 is opened without entering a proper special code, the complete health unit 10 may be disabled and will not restart unless a proper special code is thereby entered, thus preventing tampering of the health unit 10.

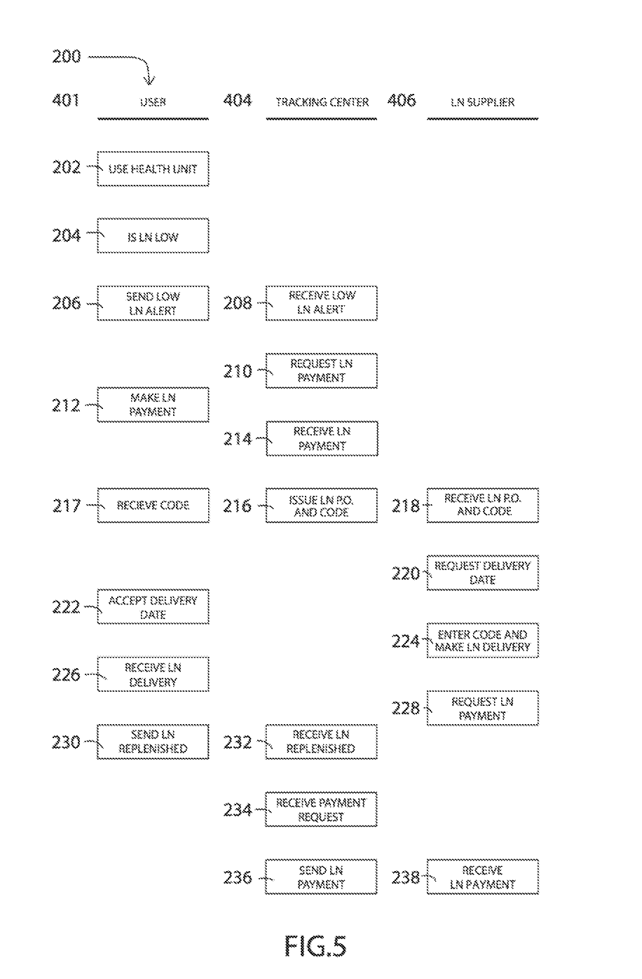

FIG. 6 is a side view of a hand held applicator unit 118 described in previous FIG. 1. To fully appreciate this embodiment this description should also be reviewed in combination with FIG. 2. Specifically, in this embodiment there may be provided an ergonomic handle 502 having a receiving end 504 and a dispensing end 506. The receiving end 504 is designed to receive tube 120 (not shown here but illustrated in FIG. 2) for delivery of the cryogenic cold nitrogen gas. Mounted on the handle 502 is the control switch 128 to turn on and off the operation of the applicator unit 118. At the dispensing end 506 there is a swivel elbow section 508 that will direct the flow of the cool nitrogen gas toward a replaceable nozzle section 130. The nozzle section 130 is illustrated to be a cone shaped nozzle, but most any shaped nozzle will work and each will have different advantages. For example, a wider cone, or small cone could be used, or a semicircular shaped cone (like half of a donut shape or any variation thereof) may work better around the eye sockets for example.

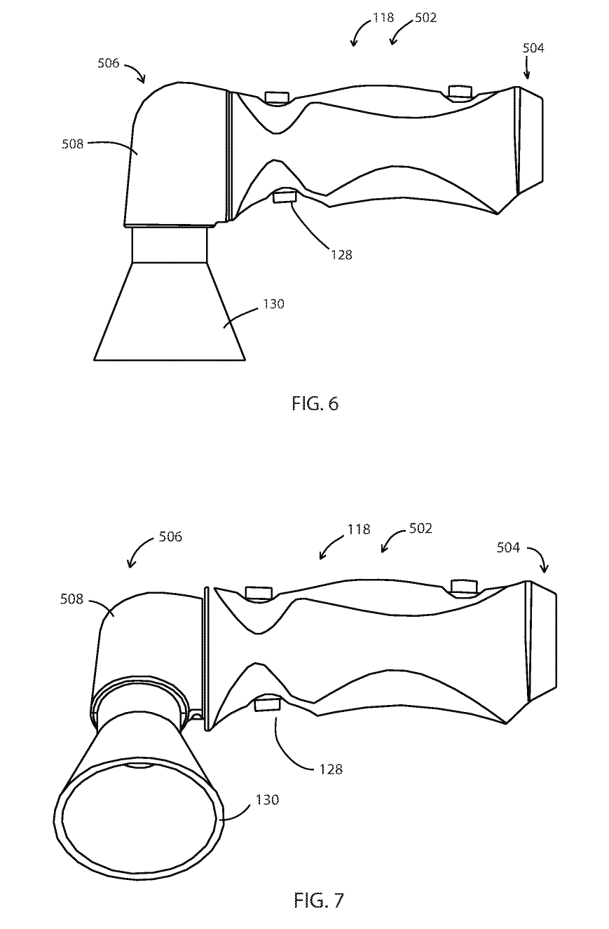

FIG. 7 is a side view of FIG. 6 with the nozzle 130 rotated about 45 degrees from that illustrated in FIG. 6. Thus, it is illustrate that the elbow 508 and nozzle 130 may in fact rotate in a circular fashion around the handle position to enable the user to conveniently reach various body parts.

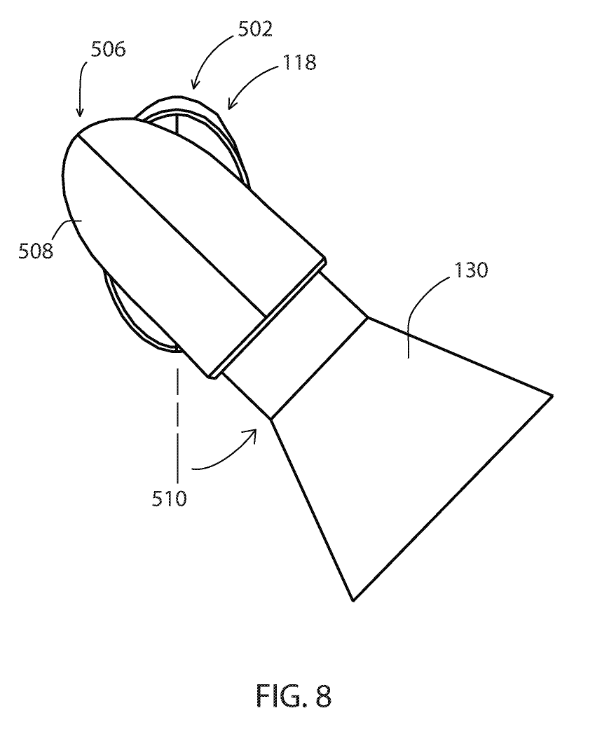

FIG. 8 is an end view of FIG. 7 illustrating the 45 deg. rotation of the nozzle. Again, it is illustrated to rotate the elbow section 508 from a centerline 510. Whereby, it is taught that the rotation may be a full 360 degrees around the centerline.

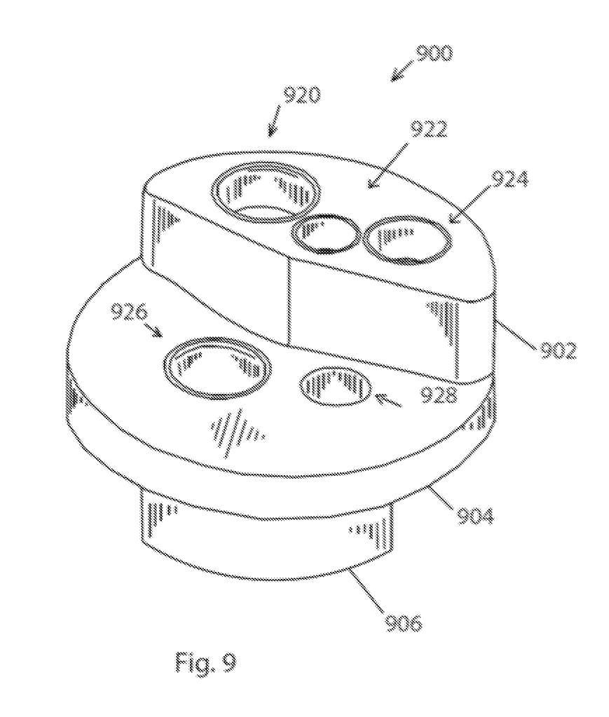

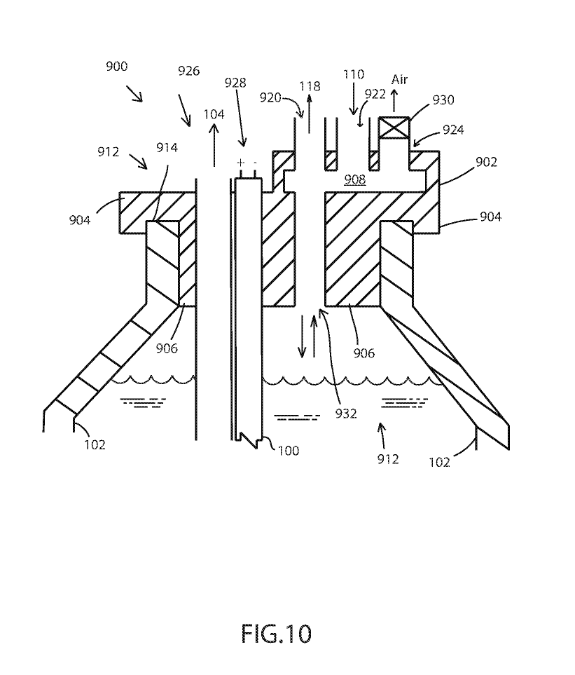

Referring now to FIGS. 9 and 10, there is illustrated one embodiment of an isometric view and cross sectional view respectively of the tank/dewar cover 900. Uniquely, in this embodiment, the tank 102 has a cover 900, which in one embodiment is taught to be made of three main sections, a chamber section 902, a plate section 904, and a sealing section 906. The sealing section 906 is designed and shaped to fit within an opening section 912 of the tank 102 and provide a pressure seal with the tank opening 912 to prevent the LN (liquid nitrogen) contents in the tank 102 from escaping therefrom. As illustrated, the sealing section 906 is shown as an elongated tubular shape, but one skilled in the art may use any shape and size that will easily mate to the tank opening to create a proper seal therebetween to prevent the leakage of the LN contents. Some alternative embodiments could include having O-rings (not shown) around the sealing section to prevent leaking of the LN while under pressure.

The plate section 904 is designed and shaped to fit over and abut to a top opening surface 914 of the tank 102 that circumnavigates the opening 912 of the tank 102. As illustrated, the plate section 904 may have a larger width than the sealing section 906.

The chamber section 902 is designed and shaped to extend above and be attached to the plate section 904. As illustrated, in one embodiment, the chamber section 902 only covers a portion of the plate section 904 top surface. Within the chamber section 902 is a chamber 908, outlets 920, 922, 924 and 932. Out let 924 includes a pressure valve 930 that will open only when the pressure in the tank 102 exceeds a predetermined safe operating level to prevent unsafe pressure build up conditions. It is noted that outlet flows are aligned so that gasified LN will go through the cover 900 with no circuitous routs, or change of directions as it passes through the cover 900.

In view of FIGS. 1, 9 and 10, one skilled in the art will realize that the LN in the tank 102 will be gasified when the heating element 100 is activated and causing the LN to be heated and thus gasified or liberated from its liquid state. The now gasified Nitrogen is then allowed to exit the tank 102 via the opening of the valve 116 and exit outlet 104 and be routed to the hand held unit 118 for localized application to a user. In one embodiment, the air compressor 110 may be operated and the valve 112a will be opened to allow the pressurized air to enter the tank 102, thus pumping the gasified Nitrogen to be forced from the tank 102 and to the hand held unit 118. In another embodiment, the air compressor 110 is not used, wherein the simple gasified or vaporized nitrogen will create pressure in the tank 102 and this pressure will be sufficient to force the nitrogen through the tubing to the hand held unit 118. In another embodiment, there is a combination of intermittently using the air compressor 110. Thus, when the vaporized nitrogen is insufficient to create sufficient flow to the hand held unit 118, the air compressor 110 will be turned on. In this operation, a sensor would be placed somewhere along the rout to the hand held unit, or placed therein, and will activate the air compressor 110, using any known technology, only when the flow is insufficient.

Similarly, upon activation of air pressure pump 110 and opening of valve 112a, atmospheric pressure within the tank 102 will increase and valve 112b will be opened to allow for LN to exit tank 102 via port 926 and through tube 115 and go to cooling chamber 104.

However, it is noted for safety reasons, if the atmospheric pressure with in the tank 102 at any time should exceed a certain level, the dangerous pressure can be relieved via the automatic opening of the safety valve 930 and allowing the excess pressure to exit via outlet 924. Any known form of a safety valve may be used herein, like one that pops open upon enough buildup of pressure on one side, and closing again when the pressure returns to acceptable limits, which such valves are well known in the art by skilled artisans.

It is noted, in another embodiment of the invention, it is contemplated elimination of the cooling chamber 104, fan 134 and associated tubing or venting. In this alternative embodiment, it is conceived to directly couple the LN exiting the tank 102 directly to the heat exchanger 106 that will have the LN flow directly therethrough the heat exchanger and thus cooling the heat exchanger 106, and routing the remaining LN and created gasified N2 back into the tank 102. Any gasified N2 will also be collected in the tank 102 and can be ported out of the tank 102 via the opening of valve 117. Thus, the use of a heat exchanger using LN filled tubing in contact with plates and fins to radiate the cold temperatures to the air flow from fan 134 will enable the cooling of the chamber 108.

Further, it should be noted that the steps described in the method of use could be carried out in many different orders according to user preference. The use of “step of” should not be interpreted as “step for”, in the claims herein and is not intended to invoke the provisions of 35 U.S.C. § 112, (6). Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

What is desired to be claimed as Letters Patent under the United States Constitution, Article 1 sec. 8, clause 8, “To promote the progress of the science and useful arts, by securing for limited times to authors and inventors the exclusive right to their respective writings and discoveries,” is set forth herein after:

1. A method of distributing liquid nitrogen (LN) to a first and second health treatment unit, with respective first and second owners at first and second addresses respectively, for exposing a user to cryotherapy temperatures, the method comprising:
a) detecting a low LN level at the first and second health treatment units;
b) sending a low LN alert from the first and second health treatment units at a tracking center;
c) requesting from the tracking center a first and second LN payment from the first and second owners respectively;
d) making a first and second LN payment by the first and second owners for replenishing LN at the first and second health treatment units respectively;
e) receiving a record of the first and second LN payments at the tracking center;
f) issue a first and second LN purchase orders to a first and second LN supplier for the first and second owners;
g) arranging a delivery date between the first and second LN supplier and the first and second owners respectively;
h) making a deliver of LN from the first and LN supplier to the first and second owners at the first and second respective addresses;
i) requesting first and second LN delivery payment by the first and second LN supplier to the tracking center;
j) detecting a full LN level at the first and second health treatment units;
k) sending a first and second LN full message from the first and second health treatment units to the tracking center indicating that the LN has been replenished at the first and second health treatment units;
l) receive the first and second LN full message at the tracking center indicating that, the first and second LN has been supplied by the first and second LN supplier to the first and second health treatment units respectively;
m) receive the request for first and second LN delivery payment from the first and second LN supplier at the tracking center; and
n) initiating a first and second LN delivery payment to the respective first and second LN supplier from the tracking center.

2. The method of claim 1, wherein the first and second LN suppliers are located at a first and second location and are owned by a single company.

3. The method of claim 2, wherein the tracking center sends a first and second unique code to the first and second health treatment units and the first and second LN suppliers respectively.

4. The method of claim 3, wherein the first and second unique code must be used by the first and second LN suppliers respectively to be able to access the first and second health treatment units.

5. A method of distributing liquid nitrogen (LN) to a first and second health treatment unit, with respective first and second owners at first and second addresses respectively, for exposing a user to cryotherapy temperatures, the method comprising:
a) detecting a low LN level at the first and second health treatment units;
b) sending a low LN alert from the first and second health treatment units at a tracking center;
c) issue a first and second LN purchase orders to a first and second LN supplier for the first and second owners respectively;
d) sending, from the tracking center, a first and second unique code to the first and second health treatment units and the first and second LN suppliers respectively; and
e) making a delivery of LN from the first and second LN supplier to the first and second health treatment units after the first and second unique codes are provided to the first and second health treatment units respectively.

6. The method of claim 5, wherein the first and second LN suppliers are located at a first and second location and are owned by a single company, and the unique code is an alpha-numeric code.

7. The method of claim 6, further comprising:
a) requesting from the tracking center a first and second LN payment from the first and second owners respectively;
h) making a first and second LN payment by the first and second owners for replenishing LN at the first and second health treatment units respectively; and
c) receiving a record of the first and second LN payments at the tracking center;

8. The method of claim 7, further comprising:
a) arranging a delivery date between the first and second LN supplier and the first and second owners respectively;
b) requesting first and second LN delivery payment by the first and second LN supplier to the tracking center;
c) detecting a full LN level at the first and second health treatment units;
d) sending a first and second LN full message from the first and second health treatment units to the tracking center indicating that the LN has been replenished at the first and second health treatment units;
e) receive the first and second LN full message at the tracking center indicating that the first and second LN has been supplied by the first and second LN supplier to the first and second health treatment units respectively;
f) receive the request for first and second LN delivery payment from the first and second LN supplier at the tracking center; and
g) initiating a first and second LN delivery payment to the respective first and second LN supplier from the tracking center.
